(19) United States
(12) Patent Application Publication
(10) Pub. No.: US 2026/0358901 A1
(43) Pub. Date: Nov. 12, 2026

(54) RING

(71) Applicant: Beijing Zitiao Network Technology Co., Ltd., Beijing (CN)

(72) Inventors: Chi Fang, Beijing (CN); Kang Cheng, Beijing (CN); Qiaokun Huang, Beijing (CN)

(21) Appl. No.: 19/573,273

(22) Filed: Mar. 20, 2026

(30) Foreign Application Priority Data
May 7, 2025 (CN) 202510586249.3

(51) Int. Cl. G06F 3/033; A61B 5/00; G06F 3/02; G06F 3/038

(52) U.S. Cl. CPC G06F 3/033; A61B 5/6826; G06F 3/0202; G06F 3/0383; G06F 2203/0331

(57) ABSTRACT

Embodiments of the disclosure provide a ring. The ring includes: a housing configured to have an annular shape to be adapted to be worn on a finger of a user and having a chamber, the chamber being adapted to accommodate a control assembly; and a button arranged on an inner side of the housing in a radial direction and coupled to the control assembly, the button being adapted to be triggered by the finger wearing the ring to cause the control assembly to implement a predetermined function. The ring design according to embodiments of the present disclosure maintains the integrity of appearance, improves utilization of the internal space, and allows the housing to use any appropriate material such as metal by placing the button inside and triggering it by pressing outside.

### CROSS-REFERENCE

This application claims priority to Chinese Patent Application No. 202510586249.3, filed May. 07, 2025, entitled “RING”, the disclosure of which is incorporated herein by reference in its entirety.

### FIELD

Example embodiments of the present disclosure generally relate to the field of wearable devices, and in particular, to a ring.

### BACKGROUND

A smart ring is a wearable electronic device that integrates a variety of sensors and communication modules and is capable of achieving multiple functions such as health monitoring, gesture control, and identity recognition. The smart ring not only provides wearing comfort and portability, but also has stronger decorative attributes and richer choices of appearance and styling.

### SUMMARY

In a first aspect of the present disclosure, a ring is provided. The ring includes: a housing configured to have an annular shape to be adapted to be worn on a finger of a user and having a chamber, the chamber being adapted to accommodate a control assembly; and a button arranged on an inner side of the housing in a radial direction and coupled to the control assembly, the button being adapted to be triggered by the finger wearing the ring to cause the control assembly to implement a predetermined function.

In some embodiments, the housing includes: an outer shell; and an inner shell arranged on a radial inner side of the outer shell and sealingly coupled to the outer shell to jointly define the chamber with the outer shell.

In some embodiments, the inner shell includes: a recess formed by recessing a predetermined depth in a radial direction at a predetermined position of the inner shell for the button to be arranged therein, and a height of the button being less than or equal to the predetermined depth.

In some embodiments, a length of the recess in a circumferential direction of the ring is greater than a length of the button, and edges of the recess in the circumferential direction have arc transitions.

In some embodiments, the button includes at least one of a mechanical button, a capacitive button, and a pressure-sensitive button.

In some embodiments, the button further includes an airbag button, and an outer surface of the button and an inner side surface of the housing are integrally formed.

In some embodiments, the inner shell further includes a through hole, the through hole being located in the recess for the button to pass through and be arranged therein.

In some embodiments, the ring further includes: a sealing member arranged between the button and the control assembly and/or between the button and an inner peripheral wall of the through hole, and being elastically deformable when the button is triggered.

In some embodiments, the sealing member further includes a covering waterproof flexible film arranged on an inner side surface of the housing to at least cover the button and the through hole.

In some embodiments, the button is located on a side of the ring close to a thumb.

In some embodiments, the control assembly includes a button unit, the button unit is coupled to the button, and the button unit is arranged in the chamber and configured to generate a press input signal in response to receiving a trigger from the user on the button.

In some embodiments, the ring further includes: a sensor at least partially arranged in the housing and coupled to the control assembly, the control assembly being configured to determine state data of the user based on user-related information acquired by the sensor.

It should be understood that the content described in this Summary section is neither intended to limit key or essential features of embodiments of the present disclosure, nor is it intended to limit the scope of the present disclosure. Other features of the present disclosure will be readily envisaged through the following description.

### BRIEF DESCRIPTION OF THE DRAWINGS

The above and other features, advantages, and aspects of embodiments of the present disclosure will become more apparent in combination with the drawings and with reference to the following detailed description. In the drawings, the same or similar reference numbers refer to the same or similar elements.

FIG. 1 shows a perspective view of a ring according to embodiments of the present disclosure;

FIG. 2 shows a perspective view of the ring according to embodiments of the present disclosure from another angle;

FIG. 3 shows a perspective view of the ring according to embodiments of the present disclosure from another angle; and

FIG. 4 shows a side view of the ring according to embodiments of the present disclosure.

### DETAILED DESCRIPTION OF EMBODIMENTS

Hereinafter, embodiments of the present disclosure will be described in more detail with reference to the drawings. Although some embodiments of the present disclosure are shown in the drawings, it should be understood that the present disclosure may be implemented in various forms and should not be construed as being limited to embodiments set forth herein. On the contrary, these embodiments are provided for a more thorough and complete understanding of the present disclosure. It should be understood that the drawings and embodiments of the present disclosure are only used for illustrative purposes, and are not used to limit the protection scope of the present disclosure.

It should be noted that any section/subsection titles provided herein are not restrictive. Various embodiments are described throughout this article, and any type of embodiment may be included under any section/subsection. In addition, embodiments described in any section/subsection may be combined with any other embodiments described in the same section/subsection and/or different sections/subsections in any manner.

In the description of embodiments of the present disclosure, the term "include/comprise" and similar terms should be understood as open-ended inclusions, that is, "include/comprise but not limited to". The term "based on" should be understood as "at least partially based on". The term "an embodiment" or "the embodiment" should be understood as "at least one embodiment". The term "some embodiments" should be understood as "at least some embodiments". Other explicit and implicit definitions may also be included below. The terms "first", "second", etc. may refer to different or same objects. Other explicit and implicit definitions may also be included below.

As a wearable device that integrates functions such as health monitoring, gesture control, and identity recognition, an interaction mode of a ring is crucial to its user experience and function implementation. A traditional smart ring may use touch for interaction, and this interaction mode requires vibration feedback to achieve a closed interaction loop. However, placing a vibration module requires a larger internal space, which reduces the space utilization inside the ring and increases the product cost. In addition, the traditional ring arranges the button on the outside of the ring and uses a separate outer ring button for interaction, which requires forming a parting line on an outer ring surface, and forming an assembly gap and a step. This method reduces the integrity of the ring, and may also destroy the color consistency of the outer surface of the ring due to production color tolerances, reducing the visual aesthetics. Therefore, there is an urgent need for a ring solution that can fully utilize the internal space of the ring and has an overall appearance.

In order to solve or at least partially solve the above problems or other potential problems of the traditional ring, embodiments of the present disclosure provide a ring. According to the solution of embodiment of the present disclosure, the button is placed inside the ring, and the button is triggered by pressing the outer surface, which ensures the integrity of the appearance of the ring and improves the utilization of the internal space of the ring. Further, the button may include a mechanical button, so that the housing has more material options, for example, the housing may be made of a metal material such as a precious metal or titanium alloy, which significantly improves the appearance and quality of the ring. At the same time, the pressing of the mechanical button provides the user with clearer mechanical feedback, which enhances the intuitiveness and reliability of the interaction. Further, the button is arranged in the recess of the ring to provide an avoidance space for the finger to press, and at the same time, a friction of the button on the skin may be avoided, and the wearing comfort is improved. Further, the button includes a sealing member, which not only ensures the pressing function of the button, but also prevents water and dust.

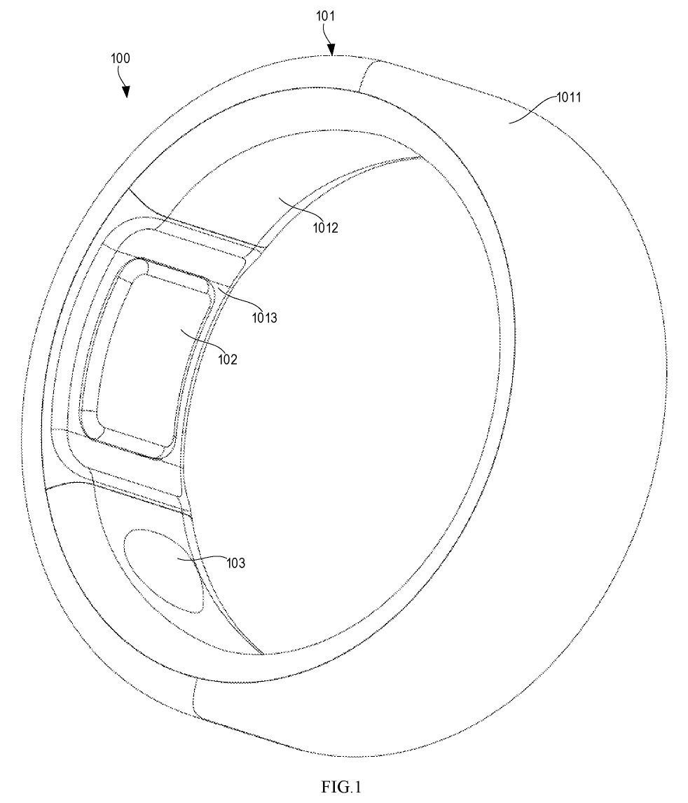

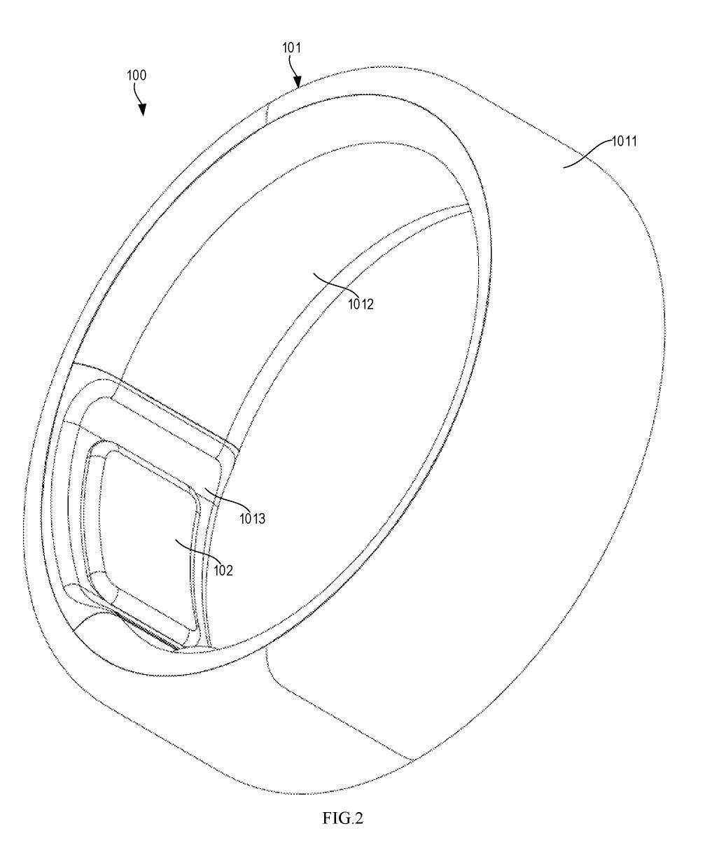

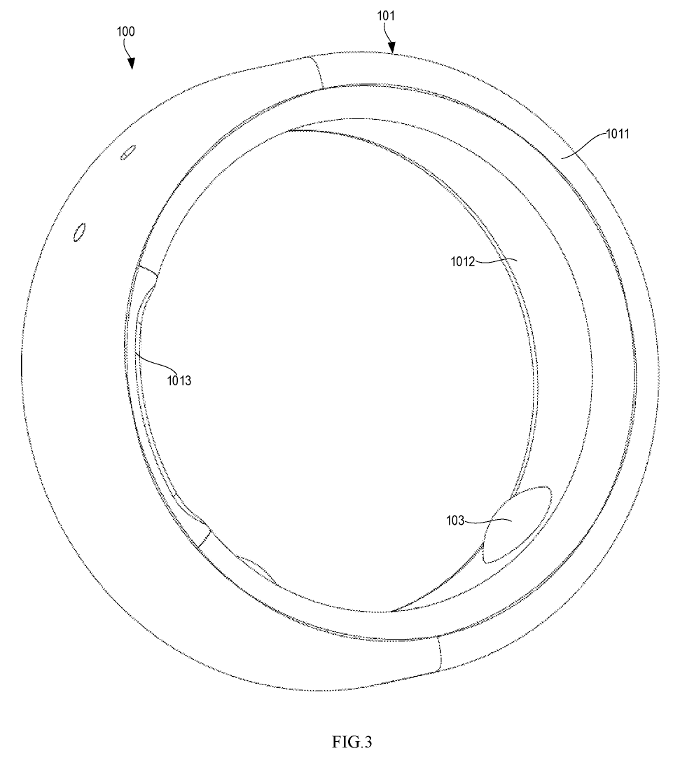

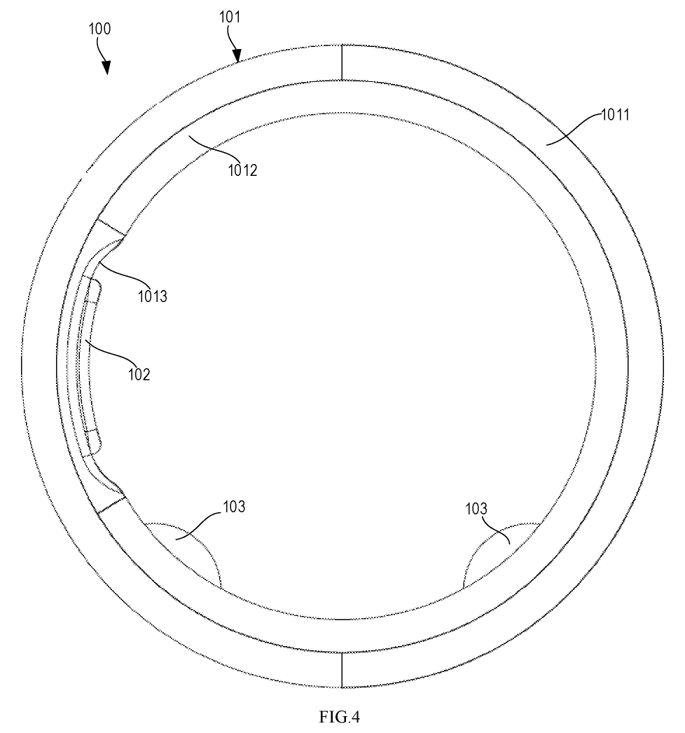

An example structure of the ring will be described below with reference to FIGS. 1-4. The ring 100 according to embodiment of the present disclosure generally includes at least a housing 101 and a button 102. The housing 101 is configured to have an annular shape so that the user may wear the ring on a finger. The housing 101 has a chamber that is substantially annular in shape for accommodating a control assembly. The control assembly may achieve multiple functions such as health monitoring, motion recording, device control, and user interaction through data acquisition, processing, transmission, and feedback.

The button 102 is arranged on an inner side of the housing 101 in a radial direction. In this way, the user may conveniently press and operate the button 102. The button 102 is coupled to the control assembly, and is triggered by the finger wearing the ring to cause the control assembly to implement a predetermined function. The user may apply an external force to a surface of the ring 100, and since the force is mutual, the external force causes the button 102 on the inner side of the ring 100 to come into contact with the finger wearing the ring, thereby triggering the button 102. In this way, the internal button 102 is triggered by external pressing, ensuring that the appearance of the ring is smooth and tidy, and improving the aesthetics of the ring 100.

For example, in some embodiments, the surface of the ring 100 may be pressed by another finger of the user, so that the button 102 on the inner side of the ring 100 is triggered by contacting with the finger wearing the ring 100. In other embodiments, the surface of the ring 100 may also be pressed by other parts of the user's body, so that the button 102 on the inner side of the ring 100 is triggered by contacting with the finger wearing the ring 100. In this way, the user does not need to precisely operate the button 102, and only needs to press the surface of the ring 100 to trigger the button 102 inside the ring 100, which makes the operation simpler and more flexible.

In some embodiments, the housing 101 may further include an outer shell 1011 and an inner shell 1012. In some embodiments, the outer shell 1011 may have a complete ring shape, and a cross section in the radial direction may have a U-shape, so as to facilitate an arrangement of the control assembly. The inner shell 1012 is arranged on a radial inner side of the outer shell 1011 and sealingly coupled to the outer shell 1011 to form the chamber.

In some embodiments, the inner shell 1012 may be formed by glue pouring and curing. Specifically, the outer shell 1011 may be first placed at an appropriate position in a mold, and the aforementioned control assembly may be placed according to a predetermined position. After the control assembly is placed in an appropriate position of the outer shell 1011, the position of each unit may be fixed by an appropriate jig, and a liquid colloid may be poured into the mold. After the colloid is solidified, appropriate grinding or trimming may be performed to form the sealed chamber and the inner shell 1012. In this way, the ring 100 may be more easily manufactured and have a better sealing performance, and the sealing and waterproof performance of the ring may be improved.

In some embodiments, the inner shell 1012 may include a recess 1013. The recess 1013 is formed by recessing a predetermined depth in a radial direction at a predetermined position of the inner shell 1012 for the button 102 to be arranged therein, and a height of the button 102 is less than or equal to the predetermined depth. In this way, a physical avoidance space may be provided for the finger on the inner side of the ring 100, the operation accuracy may be improved, and at the same time, the friction of the button on the skin may be avoided when the button 102 protrudes from an inner side surface of the ring 100, and the wearing comfort may be improved.

In some embodiments, a length of the recess 1013 in a circumferential direction of the ring 100 is greater than a length of the button 102, and edges of the recess 1013 in the circumferential direction have arc transitions. In this way, the arc transitions design makes the inner side surface of the ring 100 smoother, reduces the friction of the ring on the skin, and further improves the wearing comfort.

In some embodiments, in order to meet different needs of users, the button 102 may be of various types. The button 102 may include at least one of a mechanical button, a capacitive button, and a pressure-sensitive button. The mechanical button provides clear pressing feedback through a physical structure, and the user may clearly feel the pressing and rebounding of the button, which helps to improve the accuracy and reliability of the operation. At the same time, a physical button supports operations such as long press, short press, and light press. The user may implement multiple functions by light press, long press, short press or combination of buttons, etc. according to the needs, and the operation is more flexible. In addition, compared with a touch button, the mechanical button may provide the housing 101 with various material options, for example, the housing 101 may be made of a precious metal, titanium alloy, or the like, thereby significantly improving the appearance and quality of the ring 100.

The capacitive button triggers an operation by detecting a change in capacitance between a finger and a button surface. Since no physical structure is required, the capacitive button may be designed to be very thin, which is suitable for a thin and light smart ring. The capacitive button may also be seamlessly integrated with the surface of the ring, which maintains a simple design of the overall appearance and also has a waterproof function. The pressure-sensitive button triggers an operation by detecting the pressing force of a finger, and may perform different functions according to different pressure intensities.

In some embodiments, the button 102 may include an airbag button. The airbag button has a built-in airbag to provide a certain cushioning when pressed, improving the comfort of pressing operation. An outer surface of the button 102 and an inner side surface of the housing 101 may be integrally formed. In this way, the integrally formed design makes the inner side surface of the ring 100 smoother and tidier, improves the aesthetics and fashion sense of the product, and further improves the waterproof function.

In some embodiments, in order to facilitate an installation of the button 102, the inner shell 1012 of the ring 100 may further include a through hole. The through hole is arranged in the recess 1013 for the button 102 to pass through the through hole and be arranged in the recess 1013. In this way, the button 102 may be firmly arranged in the recess 1013.

In some embodiments, the ring 100 may further include a sealing member, which is elastically deformable when the button 102 is triggered. In some embodiments, the sealing member may be arranged between the button 102 and the control assembly. In some additional or alternative embodiments, the sealing member may be arranged between the button 102 and an inner peripheral wall of the through hole. In some embodiments, the sealing member may include a sealing ring, for example, the sealing ring may be arranged around the button 102 to improve the waterproof performance of the button 102. In this way, the sealing protection of the button 102 may be achieved, and the safety of the internal circuit and mechanical components may be ensured.

In some embodiments, the sealing member may further include a covering waterproof flexible film. The waterproof flexible film is arranged on the inner side surface of the housing 101 to form a sealed protective layer inside the housing, and at least covers the button 102 and the through hole, so as to form a seamless design between the housing 101 and the button 102. In this way, moisture and dust may be further prevented from entering an interior of the ring, and the overall sealing performance of the ring may be improved.

In some embodiments, with respect to the position of the button 102 in the circumferential direction of the ring 100, the button 102 is located on a side of the ring close to a thumb when the ring 100 is in a wearing state. In this way, the user may conveniently press and operate the button 102. In some embodiments, the control assembly includes a button unit. The button unit is arranged in the chamber and coupled to the button 102. The button unit is configured to generate a press input signal in response to receiving a trigger from the user on the button 102. In some embodiments, the press input signal may be transmitted to an external device through a communication unit, so as to provide an input for the external device to implement a corresponding function. In addition, in some embodiments, the press input signal may also be used to directly control functions such as turning on and off of a microphone unit and/or a speaker unit.

In some embodiments, the ring 100 may further include a sensor 103. The sensor 103 is at least partially arranged in the housing 101 and coupled to the control assembly. The control assembly is configured to determine state data of the user according to user-related information acquired by the sensor 103. The state data includes at least one of: heart rate, blood pressure, and blood oxygen saturation.

For example, the health sensor may include a heart rate sensor, a blood oxygen sensor, a pressure sensor, a temperature sensor, an acceleration sensor, a skin electrical reaction (EDA) sensor, and/or a bioelectrical impedance analysis (BIA) sensor, etc. The heart rate sensor may use a photoplethysmography (PPG) technology or an electrocardiogram (ECG) technology to monitor a heart rate and a heart rate variability (HRV). The blood oxygen sensor may use a PPG sensor of red light and infrared light to estimate the saturation of oxygen in the blood. The temperature sensor may measure a skin surface temperature or an internal body temperature for monitoring a body temperature change. The pressure sensor may detect a pressure change of gas or liquid for blood pressure monitoring and the like. The acceleration sensor may measure a body movement and an activity amount for motion tracking. The EDA sensor may be used to measure skin electrical activity for monitoring stress and emotional states. The BIA sensor may measure a body fat percentage and moisture by sending a small current through the body. In addition to the sensors mentioned above, the ring 100 may further include a proximity sensor, which is used to detect the presence of nearby objects for gesture recognition and contactless interaction, etc.

Various implementations of the present disclosure have been described above, and the above description is illustrative, non-exhaustive, and is not limited to the disclosed implementations. Many modifications and variations will be apparent to those of ordinary skill in the art without departing from the scope and spirit of the described implementations. The terms used herein are chosen to best explain the principles of the implementations, the practical applications, or improvements to the technology in the market, or to enable other persons of ordinary skill in the art to understand the implementations disclosed herein.

1. A ring, comprising:
a housing configured to have an annular shape to be adapted to be worn on a finger of a user and having a chamber, the chamber being adapted to accommodate a control assembly; and
a button arranged on an inner side of the housing in a radial direction and coupled to the control assembly, the button being adapted to be triggered by the finger wearing the ring to cause the control assembly to implement a predetermined function.

2. The ring of claim 1, wherein the housing comprises:
an outer shell; and
an inner shell arranged on a radial inner side of the outer shell and sealingly coupled to the outer shell to jointly define the chamber with the outer shell.

3. The ring of claim 2, wherein the inner shell comprises:
a recess formed by recessing a predetermined depth in a radial direction at a predetermined position of the inner shell for the button to be arranged therein, and a height of the button being less than or equal to the predetermined depth.

4. The ring of claim 2, wherein a length of the recess in a circumferential direction of the ring is greater than a length of the button, and edges of the recess in the circumferential direction have arc transitions.

5. The ring of claim 1, wherein the button comprises at least one of a mechanical button, a capacitive button, and a pressure-sensitive button.

6. The ring of claim 1, wherein the button comprises:
an airbag button, and an outer surface of the button and an inner side surface of the housing being integrally formed.

7. The ring of claim 3, wherein the inner shell further comprises a through hole, the through hole being located in the recess for the button to pass through and be arranged therein.

8. The ring of claim 7, wherein the ring further comprises:
a sealing member arranged between the button and the control assembly and/or between the button and an inner peripheral wall of the through hole, and being elastically deformable when the button is triggered.

9. The ring of claim 8, wherein the sealing member further comprises a covering waterproof flexible film arranged on an inner side surface of the housing to at least cover the button and the through hole.

10. The ring of claim 1, wherein the button is located on a side of the ring close to a thumb.

11. The ring of claim 1, wherein the control assembly comprises a button unit, the button unit is coupled to the button, and the button unit is arranged in the chamber and configured to generate a press input signal in response to receiving a trigger from the user on the button.

12. The ring of claim 1, further comprising:
a sensor at least partially arranged in the housing and coupled to the control assembly, the control assembly being configured to determine state data of the user based on user-related information acquired by the sensor.

13. The ring of claim 2, wherein the button comprises at least one of a mechanical button, a capacitive button, and a pressure-sensitive button.

14. The ring of claim 2, wherein the button comprises:
an airbag button, and an outer surface of the button and an inner side surface of the housing being integrally formed.

15. The ring of claim 2, wherein the button is located on a side of the ring close to a thumb.

1616 The ring of claim 7, wherein the button is located on a side of the ring close to a thumb.

17. The ring of claim 2, wherein the control assembly comprises a button unit, the button unit is coupled to the button, and the button unit is arranged in the chamber and configured to generate a press input signal in response to receiving a trigger from the user on the button.

18. The ring of claim 7, wherein the control assembly comprises a button unit, the button unit is coupled to the button, and the button unit is arranged in the chamber and configured to generate a press input signal in response to receiving a trigger from the user on the button.

19. The ring of claim 2, further comprising:
a sensor at least partially arranged in the housing and coupled to the control assembly, the control assembly being configured to determine state data of the user based on user-related information acquired by the sensor.

20. The ring of claim 7, further comprising:
a sensor at least partially arranged in the housing and coupled to the control assembly, the control assembly being configured to determine state data of the user based on user-related information acquired by the sensor.
